(19) United States
(12) Patent Application Publication
(10) Pub. No.: US 2020/0329751 A1
(43) Pub. Date: Oct. 22, 2020

(54) GENDER SPECIFIC SYNTHETIC NUTRITIONAL COMPOSITIONS COMPRISING LYCOPENE AND NUTRITIONAL SYSTEMS COMPRISING THEM

(71) Applicant: SOCIETE DES PRODUITS NESTLE S.A., Vevey (CH)

(72) Inventors: Sagar Thakkar, Singapore (SG); Francesca Giuffrida, Mezieres (CH); Frederic Destaillats, Servion (CH); Carlos Antonio De Castro, Singapore (SG); Maria-Belen Sanchez Bridge, Penthaz (CH)

(21) Appl. No.: 16/956,284

(22) PCT Filed: Dec. 20, 2018
(86) PCT No.: PCT/EP2018/086187

§ 371 Date: Jun. 19, 2020

(30) Foreign Application Priority Data
Dec. 22, 2017 (EP) 17210538.9

(51) Int. Cl. A23L 33/105; A23L 33/00; A61K 31/01

(52) U.S. Cl. CPC A23L 33/105; A61K 31/01; A23L 33/40

(57) ABSTRACT

Gender specific synthetic nutritional compositions comprising lycopene in concentrations reflecting those found in human milk produced by mothers of infants of the corresponding gender at the corresponding stage of lactation, and nutritional systems comprising them.

### TECHNICAL FIELD

The invention relates to gender specific synthetic nutritional compositions, to nutritional systems comprising them, and to their use to provide an optimised amount of lycopene to an infant.

### BACKGROUND OF THE INVENTION

Even though breastfeeding is optimal for infants, the existence of certain conditions may mean that it is contraindicated. In such cases, where the sole source of nutrition is not available to infants, alternative strategies to feed them have to be devised. Feeding infants with synthetic nutritional compositions e.g. Infant formula is one such strategy.

The compositions of the aforementioned synthetic nutritional compositions e.g. infant formulas, aim to replicate those of human milk (hereinafter HM). However, replicating HM is not a simple task. HM not only contains numerous components, its composition is extremely dynamic and these dynamic changes remain largely unexplored and uncharacterized.

The inventors have now surprisingly found that the concentration of Lycopene in HM may differ depending on the stage of lactation and the gender of a mother's infant. Because such age and gender differences in the lycopene concentration of HM have never been identified previously, these differences are not reflected in the compositions of synthetic nutritional compositions available for infants today. Given that HM is considered the gold standard with respect to infant nutrition, there remains a need for synthetic nutritional compositions tailored for infants of specific ages and genders which better reflect these identified differences.

### SUMMARY OF THE INVENTION

The invention is set out in the claims. The inventors have developed gender specific synthetic nutritional compositions for infants comprising lycopene in concentrations that reflect the concentration of lycopene found in HM produced for an infant of the same age/corresponding lactation stage and gender.

The gender specific synthetic nutritional compositions may for example, be an infant formula or a composition for an infant that is intended to be added to, or diluted with human milk.

The gender specific synthetic nutritional compositions of the invention can be prepared from a gender neutral synthetic nutritional composition by measuring out an appropriate amount of said gender neutral synthetic nutritional composition and mixing it with an additive and/or diluent e.g. lycopene and/or water.

The gender specific synthetic nutritional compositions of the invention may be included in a nutritional system. Said nutritional system may comprise a gender specific synthetic nutritional composition for a female infant and/or a gender specific composition for a male infant of the same age. A gender specific synthetic nutritional composition for a male infant may comprise more lycopene than a gender specific synthetic nutritional composition for a female infant of the same age. Said gender specific synthetic nutritional compositions may be for infants of an age selected from the group consisting of: up to 4 months of age and, 4 months of age or older.

The lycopene concentration of a gender specific synthetic nutritional composition of the invention reflects the lycopene concentration found in HM produced for an infant of the same gender and age (at a corresponding lactation stage). Because HM is considered optimal with respect to infant nutrition, a gender specific synthetic nutritional composition of the invention, and therefore a nutritional system comprising same, may provide an optimized amount of lycopene to an infant, for example an infant having the same gender as the gender to whom the synthetic nutritional composition is directed. The gender specific synthetic nutritional compositions may be used to ensure optimum lycopene intake and levels, or to prevent sub-optimal lycopene levels, and/or to optimize antioxidant capacity and/or skin health e.g. in an infant for example an infant up to 4 months of age and, 4 months of age or older.

### DETAILED DESCRIPTION

The inventors performed a longitudinal study evaluating the nutrient composition of HM collected from mothers at various stages of lactation (30 days (1 month), 60 days (2 months), and 120 days (4 months) postpartum). Surprisingly the results of this study indicated that the concentration of lycopene found in HM can differ depending on the stage of lactation and/or the gender of a mother's infant. In particular, this study indicated that the concentration of lycopene may be higher in HM produced by mothers to boys than in HM produced by mothers to girls at the same lactations stage. Details of the study, analysis techniques and results are given in example 1.

Based on the findings of the study, the inventors have designed gender specific synthetic nutritional compositions that comprise lycopene in concentrations that reflects the lycopene concentration found in HM produced for an infant of the same gender at the corresponding stage of lactation.

The term “gender specific synthetic nutritional composition” as used herein refers to any synthetic nutritional composition, intended to be consumed by an infant that is specifically adapted to the nutritional needs of either a female or male infant. Non-limiting examples of gender specific synthetic nutritional compositions for infants from birth to 4 months include; infant formulae, and a composition for infants that is intended to be added or diluted with HM e.g. HM fortifier. Non limiting examples of gender specific synthetic nutritional compositions for infants from 4 months to 12 months include infant formulae, a composition for infants that is intended to be added or diluted with HM e.g. HM fortifier, or food stuffs intended for consumption by infants either alone or in combination with HM e.g. complementary foods.

The gender specific synthetic nutritional composition is meant for consumption by an infant of the same gender as the gender for which it is specifically adapted.

The gender of an infant may be determined by their biological sex e.g. By their sex chromosomes (infants having XX sex chromosomes would be considered female and infants having XY sex chromosomes would be considered male).

The term “infant” as used herein refers to a human infant of 12 months of age or less.

In an aspect of the present invention there is provided a gender specific synthetic nutritional composition tailored for an infant comprising lycopene in a concentration reflecting that found in HM produced for an infant of the same gender at the corresponding lactation stage e.g. up to 4 months, up to 2 months, 4 months and later.

In an embodiment the gender specific synthetic nutritional composition is a male gender specific synthetic nutritional composition for an infant of up to 4 months of age and comprises lycopene in a concentration selected from the group consisting of: 0.1 to 0.95, 0.16 to 0.62 and 0.2 to 0.28 μg/mL.

The male gender specific synthetic nutritional composition may for example be for an infant of up to 2 months of age and comprises lycopene in a concentration selected from the group consisting of: 0.1 to 0.28, 0.16 to 0.24, 0.19 to 0.21 μg/mL.

The male gender specific synthetic nutritional composition may for example be for an infant of more than 2 months of age e.g. 2 to 4 months of age and may comprises lycopene in a concentration selected from the group consisting of: 0.09 to 0.96, 0.18 to 0.62, 0.27 to 0.45 μg/m L.

In an embodiment the gender specific synthetic nutritional composition is a female gender specific synthetic nutritional composition for an infant of up to 4 months of age and comprises lycopene in a concentration selected from the group consisting of: 0.07 to 0.36, 0.13 to 0.27, 0.14 to 0.22 μg/mL.

The female gender specific synthetic nutritional composition may for example be for an infant of up to 2 months of age and comprises lycopene in a concentration selected from the group consisting of: 0.07 to 0.21, 0.12 to 0.18, and 0.14 to 0.15 μg/mL.

The female gender specific synthetic nutritional composition may for example be for an infant of more than 2 months of age e.g. 2 to 4 months of age and may comprises lycopene in a concentration selected from the group consisting of: 0.11 to 0.36, 0.15 to 0.27, and 0.21 to 0.22 μg/mL.

Non-limiting examples of ages up to 4 months of age include up to 2 months of age, 2 to 3 months of age, 2 months of age and 3 months of age. Non-limiting examples of ages up to 2 months of age include; up to 2 weeks, up to 1 month, 1 month, and 2 weeks up to 1 month of age.

In an embodiment the gender specific synthetic nutritional composition is a male gender specific synthetic nutritional composition for an infant of 4 months of age and older and comprises lycopene in a concentration selected from the group consisting of: 0.06 to 0.39, 0.1 to 0.22, and 0.15 to 0.18 μg/mL.

In an embodiment the gender specific synthetic nutritional composition is a female gender specific synthetic nutritional composition for an infant of 4 months of age and older and comprises lycopene in a concentration selected from the group consisting of: 0.09 to 0.27, 0.1 to 0.2, 0.12 to 0.16 μg/mL.

Non limiting examples of an age 4 months of age and older include; 4, 5, 6, 7, 8, 9, 10, 11, and 12 months of age, 4 to 6 months of age, 4 to 12 months of age, 6 to 12 months of age, 6 to 9 months of age, and 9 to 12 months of age.

The lycopene concentration of the gender specific synthetic nutritional compositions defined herein is expressed in μg/mL. This may refer to the lycopene concentration of a reconstituted gender specific synthetic nutritional composition.

The lycopene concentration of a composition can be measured by methods well known in the art. In particular, the lycopene concentration can be measured by extraction of the lipids and lipophilic molecules by organic solvents. The analytical measurement of these extracted molecules may be done in two steps. The first step is chromatographic separation by HPLC followed by second step of detection by diode array detectors and UV detectors.

Any form of lycopene suitable for administration to an infant to whom the gender specific synthetic nutritional composition is directed may be comprised within in the gender specific synthetic nutritional compositions of the invention. lycopene may for example be added as free lycopene and/or an ester of lycopene e.g. as one or more fatty ester acid of lycopene.

The lycopene, in any form it is used e.g. free lycopene or an ester of lycopene, may stem from natural sources, in particular it may stem from animal or plant or from algae sources.

The gender specific synthetic nutritional compositions of the invention can also comprise any other ingredients or excipients known to be employed in the type of gender specific synthetic nutritional composition in question e.g. infant formula.

Non-limiting examples of such ingredients include: proteins, amino acids, carbohydrates, oligosaccharides, lipids, prebiotics or probiotics, essential fatty acids, nucleotides, nucleosides, vitamins, minerals and other micronutrients.

Non limiting examples of proteins include: casein, alpha-lactalbumin, whey, soy protein, rice protein, corn protein, oat protein, barley protein, wheat protein, rye protein, pea protein, egg protein, sunflower seed protein, potato protein, fish protein, meat protein, lactoferrin, serum albumin, immunoglobins, and combinations thereof.

Non limiting examples of amino acids include leucine, threonine, tyrosine, Isoleucine, arginine, alanine, histidine, isoleucine, proline, valine, cysteine, glutamine, glutamic acid, glycine, serine, arginine, lysine, methionine, phenylalanine, tryptophane, asparagine, aspartic acid, and combinations thereof.

Non-limiting examples of carbohydrates include lactose, saccharose, maltodexirin, starch, and combinations thereof.

Non-limiting examples of lipids include: palm olein, high oleic sunflower oil, high oleic safflower oil, canola oil, fish oil, coconut oil, bovine milk fat, and combinations thereof.

Non-limiting examples of essential fatty acids include: linoleic acid (LA), α-linolenic acid (ALA) and polyunsaturated fatty acids (PUFAs). The gender specific synthetic nutritional compositions of the invention may further contain gangliosides monosialoganglioside-3 (GM3) and disialogangliosides 3 (GD3), phospholipids such as sphingomyelin, phospholipids phosphatidylcholine, phosphatidylethanolamine, phosphatidylinositol, phosphatidylserine, and combinations thereof.

None limiting examples of prebiotics include: oligosaccharides optionally containing fructose, galactose, mannose; dietary fibers, in particular soluble fibers, soy fibers; inulin; and combinations thereof. Preferred prebiotics are fructo-oligosaccharides (FOS), galacto-oligosaccharides (GOS), isomalto-oligosaccharides (IMO), xylo-oligosaccharides (XOS), arabino-xylo oligosaccharides (AXOS), mannan-oligosaccharides (MOS), oligosaccharides of soy, glycosylsucrose (GS), lactosucrose (LS), lactulose (LA), palatinose-oligosaccharides (PAO), malto-oligosaccharides, gums and/or hydrolysates thereof, pectins and/or hydrolysates thereof, and combinations of the foregoing.

Further examples of oligosaccharide are described in Wrodnigg, T. M.; Stutz, A. E. (1999) Angew. Chem. Int. Ed. 38:827-828 and in WO 2012/069416 which is incorporated herein by reference.

Non limiting examples of probiotics include: Bifidobacterium, Lactobacillus, Lactococcus, Enterococcus, Streptococcus, Kluyveromyces, Saccharoymces, Candida, in particular selected from the group consisting of Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Saccharomyces cerevisiae, Saccharomyces boulardii or mixtures thereof, preferably selected from the group consisting of Bifidobacterium longum NCC3001 (ATCC BAA-999), Bifidobacterium longum NCC2705 (CNCM I-2618), Bifidobacterium longum NCC490 (CNCM 1-2170), Bifidobacterium lactis NCC2818 (CNCM 1-3446), Bifidobacterium breve strain A, Lactobacillus paracasei NCC2461 (CNCM 1-2116), Lactobacillus johnsonii NCC533 (CNCM 1-1225), Lactobacillus rhamnosus GG (ATCC53103), Lactobacillus rhamnosus NCC4007 (CGMCC 1.3724), Enterococcus faecium SF 68 (NCC2768; NCIMB10415), and combinations thereof.

Non-limiting examples of Nucleotides include: cytidine monophosphate (CMP), uridine monophosphate (UMP), adenosine monophosphate (AMP), guanosine monophosphate (GMP), and combinations thereof.

Non-limiting examples of vitamins and minerals include: vitamin E, vitamin B1, vitamin B2, vitamin B6, vitamin B12, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, L-carnitine, and combinations thereof. Minerals are usually added in salt form.

Other suitable and desirable ingredients of synthetic nutritional compositions, that may be employed in the gender specific synthetic nutritional compositions of the invention are described in guidelines issued by the Codex Alimentarius with respect to the type of synthetic nutritional composition in question e.g. Infant formula, HM fortifier, follow on formula, or food stuffs intended for consumption by infants e.g. complementary foods.

The gender specific synthetic nutritional compositions of the invention may be prepared by methods well known in the art for preparing the type of gender specific synthetic nutritional composition in question e.g. infant formulae, follow on formulae, a composition for infants that is intended to be added or diluted with HM e.g. HM fortifier, or food stuffs intended for consumption by infants either alone or in combination with HM e.g. complementary foods.

An exemplary method for preparing a gender specific powdered infant formula is as follows. A protein source, carbohydrate source, and fat source may be blended together in appropriate proportions. Emulsifiers maybe included in the blend. Vitamins, minerals and lycopene may be added at this point (for example in a vitamin premix containing lycopene) but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture.

The liquid mixture may then be thermally treated to reduce bacterial loads. For example, the liquid mixture may be rapidly heated to a temperature in the range of about 80° C. to about 110° C. for about 5 seconds to about 5 minutes. This may be carried out by steam injection or by heat exchanger; for example a plate heat exchanger.

The liquid mixture may then be cooled to about 60° C. to about 85° C.; for example by flash cooling. The liquid mixture may then be homogenised; for example in two stages at about 7 MPa to about 40 MPa in the first stage and about 2 MPa to about 14 MPa in the second stage. The homogenised mixture may then be further cooled to add any heat sensitive components such as vitamins and minerals. The pH and solids content of the homogenised mixture is conveniently standardised at this point.

The homogenised mixture can be transferred to a suitable drying apparatus such as a spray drier or freeze drier and converted to powder. The powder should have a moisture content of less than about 3% by weight.

If it is desired probiotic(s) can be added, they may be cultured according to any suitable method and prepared for addition to the infant formula by freeze-drying or spray-drying for example. Alternatively, bacterial preparations can be bought from specialist suppliers such as Christian Hansen and Morinaga already prepared in a suitable form for addition to food products such as infant formula. Such bacterial preparations may be added to the gender specific powdered infant formula by dry mixing.

The gender specific synthetic nutritional compositions of the invention may also be prepared from a gender neutral synthetic nutritional composition in a method comprising; measuring out an appropriate amount of said gender neutral synthetic nutritional composition and mixing it with an additive and/or a diluent e.g. lycopene and/or water so as to arrive at a gender specific synthetic nutritional composition in accordance with the invention. The additive may be a gender specific additive.

The additive may be a gender specific additive comprising lycopene in a particular concentration so that when mixed with the gender neutral synthetic nutritional composition, and optionally a diluent, the resulting mixture is a gender specific synthetic nutritional composition in accordance with the invention.

The gender neutral synthetic nutritional composition can be prepared by methods well known in the art for the type of composition in question e.g. as laid out above for infant formula.

The term “gender neutral” as used herein is synonymous with unisex.

One or more of the gender specific synthetic nutritional compositions of the invention can be included in a nutritional system.

The term “nutritional system” as used herein refers to a collection of more than one synthetic nutritional composition advertised or sold as part of the same product range e.g. a collection of infant formulas sold under the same brand and adapted/tailored to the nutritional needs of infants of differing ages and/or genders and/or delivered by different methods e.g. C-section. The synthetic nutritional compositions making up the nutritional system may be packaged individually e.g. in capsules or boxes. Said packages can be sold individually, grouped together e.g. wrapped by plastic film or combined in a box, or in a combination of these two ways. The nutritional system may also comprise synthetic nutritional compositions for children older than 12 months.

In a further aspect of the present invention there is provided a nutritional system comprising a gender specific synthetic nutritional composition of the invention.

In an embodiment the nutritional system comprises a gender specific synthetic nutritional composition for a male infant and a gender specific synthetic nutritional composition for a female infant wherein, said male and female gender specific synthetic nutritional compositions are for infants of the same age and wherein the concentration of lycopene in said gender specific synthetic nutritional composition for a male infant is higher from that in said gender specific synthetic nutritional composition for a female infant.

The concentration of lycopene in said male gender synthetic nutritional compositions may be higher by any amount.

In an embodiment, the nutritional system comprises a gender specific synthetic nutritional composition for a male infant up to 4 months of age, and a gender specific synthetic nutritional composition for a female infant up to 4 months of age wherein, the concentration of lycopene in said male gender specific synthetic nutritional composition is higher than the lycopene concentration of said female gender specific synthetic nutritional composition

Said male gender specific synthetic nutritional composition may comprise for example 0.03 to 0.84, 0.05 to 0.22, 0.06 to 0.2 μg/mL more lycopene than the female gender specific synthetic nutritional composition.

The gender specific synthetic nutritional compositions may for example be for an infant of up to 2 months of age and the male gender specific synthetic nutritional composition may comprises 0.03 to 0.2, 0.05 to 0.07 μg/mL more lycopene than the female gender specific synthetic nutritional composition.

The gender specific synthetic nutritional compositions may for example be for an infant of more than 2 months of age for example 2 to 4 months of age and the male gender specific synthetic nutritional composition may comprises 0.05 to 0.84, 0.05 to 0.24 μg/mL more lycopene than the female gender specific synthetic nutritional composition.

In yet another specific embodiment the nutritional system comprises a gender specific synthetic nutritional composition for a male infant of from 4 months of age, and a gender specific synthetic nutritional composition for a female infant of from 4 months of age wherein, the concentration of lycopene in said male gender specific synthetic nutritional composition is higher than the lycopene concentration of said female gender specific synthetic nutritional composition.

The concentration of lycopene in said female gender synthetic nutritional compositions may be higher by any amount.

Said male gender specific synthetic nutritional composition may comprise for example 0.01 to 0.3, 0.04 to 0.12, and 0.01 to 0.032 μg/mL more lycopene than the female gender specific synthetic nutritional composition.

The nutritional system of the invention may also comprise nutritional compositions for children older than 12 months.

Gender specific synthetic nutritional compositions according to the invention are particularly suitable for use in a method of preparing single servings of infant formula using capsules, each capsule of which contains a unit dose of a synthetic nutritional composition e.g. a gender specific synthetic nutritional composition in a concentrated form, and which is equipped with opening means contained within the capsule to permit draining of the reconstituted synthetic nutritional composition directly from the capsule into a receiving vessel such as a baby bottle. Such a method is described in WO2006/077259.

The different synthetic nutritional compositions, including synthetic nutritional compositions tailored for an infant of a specific age and/or genders, may be packed into individual capsules and presented to the consumer in multipacks containing a sufficient number of capsules to meet the requirements of an infant of a particular age/age range and/or gender, for one week for example. Suitable capsule constructions are disclosed in WO2003/059778.

The capsules can contain the synthetic nutritional compositions, (gender specific and gender neutral) in the form of powders or concentrated liquids in both cases for reconstitution by an appropriate amount of water. Both the composition and the quantity of infant formula in the capsules may vary according to the gender and/or age of the infant. If necessary, different sizes of capsules may be provided for the preparation of infant formulas for infants of different genders and/or ages.

Because HM is the gold standard when it comes to infant nutrition, and because the lycopene concentration of the gender specific synthetic nutritional compositions of the invention better reflect the lycopene concentration found in HM at the corresponding lactation stage for mothers of infants of the corresponding gender, they, and the nutritional systems comprising them, may be used to provide an optimum amount of lycopene to an infant and to ensure optimum lycopene levels or to prevent or treat sub-optimal lycopene levels and/or to optimize antioxidant capacity and/or skin health in an infant e.g. an infant up to 4 months of age or an infant 4 months of age and older.

Lycopene has many health benefits which include its antioxidant capacity, benefits on protection of skin from photodamage.

In another aspect of the present invention there is provided a gender specific synthetic nutritional composition of the invention for use to prevent and/or treat sub-optimal lycopene levels e.g. in an infant e.g. an infant up to 4 months of age, or 4 months of age or older.

The gender specific synthetic nutritional compositions of the invention may provide an optimum amount of lycopene to an infant, in particular to an infant up to 4 months of age, or 4 months of age or older.

The nutritional system may for example provide an optimum amount of lycopene to an infant, in particular for an infant up to 12, 11, 10, 9, 8, 7, 6, 5, 4, 3, 2, 1 months of age and/or up to 2 weeks of age.

In another aspect of the present invention there is provided a method for providing an optimum amount of lycopene and/or preventing or treating a sub-optimal lycopene level, and/or optimizing antioxidant capacity and or skin health in an infant comprising:
a) Optionally preparing a gender specific synthetic nutritional composition of the invention from a gender-neutral synthetic nutritional composition;
b) Feeding a gender specific synthetic nutritional composition according to the invention to an infant, in particular an infant of the corresponding gender and age, in particular an infant of up to 4 months of age, or 4 months of age or older.

In another aspect of the present invention there is provided the use of a composition of the invention in the manufacture of a composition to providing an optimum amount of lycopene and/or to prevent or treat a sub-optimal lycopene level, and/or to optimize antioxidant capacity and or skin health in an infant e.g. an infant up to 4 months of age, or 4 months of age or older.

As stated herein. The gender specific synthetic nutritional compositions may be prepared from gender neutral synthetic nutritional compositions. Accordingly, in another aspect of the present invention there is provided a kit for providing an optimized amount of lycopene to an infant, in particular an infant up to 4 months of age, or 4 months of age or older, the kit comprising:
a) A gender neutral synthetic nutritional composition
b) A label indicating dosage requirements for an infant so as to arrive at a gender specific nutritional composition in accordance with the invention.

The dosage requirements may be with respect to the quantity of the gender neutral synthetic nutritional employed and/or consumption frequency e.g. 4 times per day.

It should be appreciated that all features of the present invention disclosed herein can be freely combined and that variations and modifications may be made without departing from the scope of the invention as defined in the claims. Furthermore, where known equivalents exist to specific features, such equivalents are incorporated as if specifically referred to in this specification.

There now follows a series of non-limiting examples that serve to illustrate the invention.

### EXAMPLES

### Example 1

Longitudinal Clinical Trial:

The present inventors designed a longitudinal clinical trial with 50 lactating mothers with milk sampling at 30 (visit 1), 60 (visit 2) and 120 (visit 3) days post-partum. The milk samples were quantitatively analyzed for lycopene.

Human Milk Collection:

The protocol and collection of human milk was reviewed and approved by the local ethical committee of Singapore. The study took place at National University of Singapore. Volunteer mothers of term infants, who were apparently healthy and non-smokers (n=50; 31.1±3.1-year old) provided breast milk samples (approximately 30 mL). Samples were collected after full expression from one breast using a milk pump, while the baby was fed on the other breast. All efforts were made to collect complete feed that included fore-milk, mid-milk and hind-milk as a representation of one feed, to avoid within feed variation of lipid content. Approximately 30 mL aliquot was separated in a conical polypropylene tube for this study and the rest was fed to the infant. Samples collected for research were stored at −80° C. until analyses. Data collection points were 30 days (1 month), 60 days (2 months) and 120 days (4 months) postpartum.

Measurement of the Lycopene Concentrations in Samples:

The lycopene concentration of each sample was measured via a method employing the following 3 steps: by

### Step 1. Liquid-Liquid Extraction:

The following were added to 1 Ml of a human milk sample:
5 μL of EtOH/BHT at 79 g/L,
10 μL of deferoxamine mesylate,
4 mL of MeOH and,
1 mL of KOH 30% w:w.

The composition was Mix vigourously for 30 seconds using a vortex, then placed for 30 minutes in a 37° C. waterbath for saponification. After the saponification, the composition was cooled down on ice. After cooling, 5 mL of hexane/BHT (4.1.3) was added to the and the composition was mixed using a vortex for 20 seconds. The composition was centrifuged at 2500 rpm/min for 10 minutes at 4° C. The organic phase was collected in a 15 mL pyrex tube. The above was repeated two additional times with human milk taken from the same sample. All organic phases collected from the same sample were pooled (with the previous ones).

Step 2. Evaporation and Resuspension

The organic phases were placed under nitrogen gas flow until complete dryness. The deposit was then Redissolved in 70 μL of dioxane/ethanol and mixed with a vortex for 15 seconds. 70 μL of acetonitrile was added to the composition and the composition was mixed with a vortex for 15 seconds.

If a precipitate formed, the composition was centrifuged at 2500 rpm/min for 10 minutes at room temperature. The complete volume of the tube containing the resuspended composition was transferred to the UPLC.

Step 3. Chromatorgraphy & UV Detection

ACQUITY UPLC system with Fluorescence Detector and UV Detector.

ACQUITY UPLC HSS T3 Column, 100 Å, 1.8 μm, 2.1 mm Waters X 150 mm, 176001133.

Mobile phase A: Ammonium acetate 0.05M (In a 1-L bottle, dissolve 3.85 g of ammonium acetate into 1000 mL of water).

Mobile phase B: ACN/Ether/MeOH (In a 1-L bottle, weight 588.75 g of acetonitril, 71.34 g of ether diethylic and 118.77 g of methanol).

The following conditions were used for the chromatography:

| Time (min) | Flow (mL/min) | % A | % B | Curve |
|---|---|---|---|---|
| 0 | 0.4 | 25 | 75 | |
| 20 | 0.4 | 25 | 75 | 6 |
| 22 | 0.4 | 22 | 78 | 6 |
| 22.1 | 0.4 | 20 | 80 | 6 |
| 30 | 0.4 | 0 | 100 | 6 |
| 42 | 0.4 | 0 | 100 | 6 |
| 42.1 | 0.4 | 25 | 75 | 6 |
| 55 | 0.4 | 25 | 75 | 6 |

The results of the analysis of the HM, with respect to the lycopene concentration, are shown in tables 1a and 1 b.

TABLE 1a

| | | Lycopene Concentration μg/mL | | | | | |
|---|---|---|---|---|---|---|---|
| | | Female | | | | | |
| Stage | Min | Median | mean | Max | SD | QT1 | QT3 |
| 30 | 0.078969 | 0.14106792 | 0.146162 | 0.203153 | 0.044323 | 0.129621 | 0.175448 |
| days | | | | | | | |
| 60 | 0.116476 | 0.21660918 | 0.218672 | 0.355545 | 0.086619 | 0.15448 | 0.266559 |
| days | | | | | | | |
| 120 | 0.095762 | 0.12438851 | 0.158726 | 0.263428 | 0.070631 | 0.111314 | 0.198736 |
| days | | | | | | | |

TABLE 1b

| | | Lycopene Concentration μg/mL | | | | | |
|---|---|---|---|---|---|---|---|
| | | Male | | | | | |
| Stage | Min | Median | Mean | Max | SD | QT1 | QT3 |
| 30 | 0.111077 | 0.20342295 | 0.196345 | 0.273456 | 0.053958 | 0.053958 | 0.233657 |
| days | | | | | | | |
| 60 | 0.094309 | 0.27653323 | 0.441849 | 0.954705 | 0.453395 | 0.453395 | 0.615619 |
| days | | | | | | | |
| 120 | 0.06436 | 0.15616268 | 0.178047 | 0.381967 | 0.10645 | 0.10645 | 0.219545 |
| days | | | | | | | |

Statistical analysis: the results of the compositional analysis were then subject to a statistical analysis employing the following statistical model:

Lycopene=B₀+B₁age+B₂age²+B₃sex+B₄age*sex+B₅age²*sex+ε

Age is represented in both linear and quadratic terms and is measured in days·ε refers to the random effect of the model which controls for within subject variability.

The different suffixes (B₀, B₁, B₂ . . . ) represent the different estimated slopes attached to the corresponding variable (age, linear and quadratic, sex and/or their interaction).

Table II shows the estimates for timeframe differences along with the corresponding Pvalues.

The results of the Statistical analysis (statistical inference) are show in table II.

TABLE II
Example 2

| Timeframe | Variable | Estimate | SE | Pvalue | Unit |
|---|---|---|---|---|---|
| 30 days | lycopene | 0.01502 | 0.07110 | 0.83273 | μg/mL |
| 60 days | lycopene | 0.21352 | 0.08710 | 0.01424 | μg/mL |
| 90 days | lycopene | 0.22323 | 0.09830 | 0.02315 | μg/mL |
| 120 days | lycopene | 0.04414 | 0.04414 | 0.53360 | μg/mL |

Examples of gender specific synthetic nutritional compositions (infant formulas) tailored to infants of up to 4 months of age and from 4 months of age are given in table III. In combination these are an example of a nutritional system of the invention.

TABLE III

| | Up to 4 months of age | | 4 months of age and older | |
|---|---|---|---|---|
| | M | F | M | F |
| Ingredients | Per Liter | | Per Liter | |
| Energy (kcal) | 670 | 670 | 630 | 630 |
| Protein (g) | 12.1 | 12.1 | 11.3 | 11.3 |
| Fat (g) | 35.649 | 35.649 | 31.446 | 31.458 |
| Linoleic | 5.3 | 5.3 | 4.7 | 4.7 |
| acid (g) | | | | |
| α-Linolenic | 675 | 675 | 600 | 600 |
| acid (mg) | | | | |
| Lactose (g) | 74.7 | 74.7 | 75 | 75 |
| Prebiotic | 4.3 | 4.3 | 4.0 | 4.0 |
| (100% GOS) (g) | | | | |
| Lycopene | 270 | 210 | 160 | 130 |
| (μg) | | | | |
| Minerals (g) | 2.5 | 2.5 | 2.3 | 2.3 |
| Na (mg) | 150 | 150 | 158 | 158 |
| K (mg) | 590 | 590 | 504 | 504 |
| Cl (mg) | 430 | 430 | 410 | 410 |
| Ca (mg) | 410 | 410 | 378 | 378 |
| P (mg) | 210 | 210 | 208 | 208 |
| Mg (mg) | 50 | 50 | 44 | 44 |
| Mn (μg) | 50 | 50 | 32 | 32 |
| Se (μg) | 13 | 13 | 19 | 19 |
| Vitamin A | 820 | 770 | 800 | 720 |
| (μg RE) | | | | |
| Vitamin D | 10 | 10 | 9.5 | 9.5 |
| (μg) | | | | |
| Vitamin E | 5.4 | 5.4 | 5.0 | 5.0 |
| (mg TE) | | | | |
| Vitamin K1 | 54 | 54 | 50 | 50 |
| (μg) | | | | |
| Vitamin C (mg) | 67 | 67 | 95 | 95 |
| Vitamin B1 (mg) | 0.47 | 0.47 | 0.6 | 0.6 |
| Vitamin B2 (mg) | 1 | 1 | 0.6 | 0.6 |
| Niacin (mg) | 6.7 | 6.7 | 3.2 | 3.2 |
| Vitamin B6 (mg) | 0.5 | 0.5 | 0.4 | 0.4 |
| Lactoferrin | 1 | 1 | 0.3 | 0.3 |
| (bovine) g | | | | |
| Folic acid | 60 | 60 | 95 | 95 |
| (μg) | | | | |
| Pantothenic | 3 | 3 | 5.0 | 5.0 |
| acid (mg) | | | | |
| Vitamin B12 | 2 | 2 | 1.3 | 1.3 |
| (μg) | | | | |
| Biotin (μg) | 15 | 15 | 12.6 | 12.6 |
| Choline (mg) | 67 | 67 | 95 | 95 |
| Fe (mg) | 8 | 8 | 6.3 | 6.3 |
| I (μg) | 100 | 100 | 95 | 95 |
| Cu (mg) | 0.4 | 0.4 | 0.4 | 0.4 |
| Zn (mg) | 5 | 5 | 5.7 | 5.7 |

1-2. (canceled)

3. A gender specific synthetic nutritional composition wherein, if the concentration of lycopene is tailored to a male infant of up to 4 months of age it is within the range of 0.1 to 0.95 μg/mL and, if the concentration of lycopene is tailored to a female infant of up to 4 months of age it is within the range of 0.07 to 0.36 μg/mL; if the concentration of lycopene is tailored to a male infant of 4 months of age or older it is within the range of 0.06 to 0.39 μg/mL and, if the concentration of lycopene is tailored to a female infant of 4 months of age or older it is within the range of 0.09 to 0.27 μg/mL.

4. A gender specific synthetic nutritional composition according to claim 3 wherein, the gender specific synthetic nutritional composition is selected from the group consisting of: infant formula, and a composition for infants that is intended to be added to or diluted with human milk.

5-12. (canceled)

13. A method for providing an optimum amount of lycopene to an infant comprising:
a. Feeding a gender specific synthetic nutritional composition wherein, if the concentration of lycopene is tailored to a male infant of up to 4 months of age it is within the range of 0.1 to 0.95 μg/mL and, if the concentration of lycopene is tailored to a female infant of up to 4 months of age it is within the range of 0.07 to 0.36 μg/mL, if the concentration of lycopene is tailored to a male infant of 4 months of age or older it is within the range of 0.06 to 0.39 μg/mL and, if the concentration of lycopene is tailored to a female infant of 4 months of age or older it is within the range of 0.09 to 0.27 μg/mL to an infant in need of same.

14. A method as defined in claim 13 for use to prevent or treat sub-optimal lycopene levels and promote skin health in an infant.

15. A kit for providing an optimized amount of lycopene to an infant, the kit comprising:
a. A gender neutral synthetic nutritional composition
b. A label indicating dosage requirements for an infant so as to arrive at a gender specific synthetic nutritional composition wherein, if the concentration of lycopene is tailored to a male infant of up to 4 months of age it is within the range of 0.1 to 0.95 μg/mL and, if the concentration of lycopene is tailored to a female infant of up to 4 months of age it is within the range of 0.07 to 0.36 μg/mL; if the concentration of lycopene is tailored to a male infant of 4 months of age or older it is within the range of 0.06 to 0.39 μg/mL and, if the concentration of lycopene is tailored to a female infant of 4 months of age or older it is within the range of 0.09 to 0.27 μg/mL.
